(19) United States
(12) Patent Application Publication
(10) Pub. No.: US 2022/0107377 A1
(43) Pub. Date: Apr. 7, 2022

(54) Magnetic Resonance Imaging Method Of Generating And Displaying Quantitative T1-Weighted Subtraction Maps (dt1 "delta T1" Maps)

(71) Applicant: Imaging Biometrics, LLC, Elm Grove, WI (US)

(72) Inventors: Kathleen Schmainda, Elm Grove, WI (US); Todd Jensen, Shorewood, WI (US)

(73) Assignee: Imaging Biometrics, LLC, Elm Grove, WI (US)

(21) Appl. No.: 17/494,791

(22) Filed: Oct. 5, 2021

Related U.S. Application Data
(60) Provisional application No. 63/088,295, filed on Oct. 6, 2020.

(51) Int. Cl. G01R 33/50; G01R 33/56; G06N 20/00

(52) U.S. Cl. CPC G01R 33/50; G06N 20/00; G01R 33/5601; G01R 33/5608

(57) ABSTRACT

A method of generating and displaying quantitative T1-weighted subtraction images or maps, also known as delta T1 maps, using a “FLOW” (red/blue) color look up table (CLUT) automatically without additional processing steps or manual intervention, The delta T1 weighted maps display objective visualization via red image enhancement and spatial visualization of underlying anatomy via computational generation of small values of ‘blue’ non-enhancing regions.

### CROSS-REFERENCE TO RELATED APPLICATIONS

This application claims priority to U.S. Provisional Patent Application No. 63/088,295, filed on Oct. 6, 2020, the entire disclosure of which is incorporated herein by reference.

### BACKGROUND OF THE INVENTION

Magnetic resonance imaging (MRI) relies on the relaxation properties of excited hydrogen nuclei in water and in lipids to create images. When a target object to be imaged is placed in a uniform magnetic field, the forces in the magnetic field cause the spins of atomic nuclei having a non-zero spin to align in a particular manner with the applied magnetic field. By way of example, hydrogen atoms have a simple spin (1/2) and therefore align either parallel or antis-parallel (parallel but oppositely directed or oriented) to the magnetic field. A radio frequency pulse (RF) is then applied in a direction perpendicular to the magnetic field and removed. When the RF signal is removed, the atomic nuclei relax. During the relaxation process, the nuclei release energy by emitting an RF signal unique to the nuclei, which may be measured by a conductive field coil placed around the target object. This measurement is processed or reconstructed to obtain the magnetic resonance images.

The signal intensity of a given tissue type depends upon the density of the protons in the tissue. However, the contrast of the image, or the difference in luminance or color that distinguishes one object from another in the same field of view also depends on two other tissue-specific parameters: the longitudinal relaxation time (T1) and the transverse relaxation time (T2). T1 defines the time required for the displaced nuclei to return to equilibrium, that is to say, the time required for the nuclei to realign themselves in the magnetic field. T2 is the time required for the signal emitted by a specific tissue type to decay, or stated differently, T2 is the time constant for the decay of transverse magnetization arising from natural atomic and molecular interactions within the target object. However, in practice, the observed decay rate of transverse magnetization is faster than the mathematically predicted decay rate as a result of inhomogeneities in the magnetic field caused by magnet defects or magnetic field distortions. The observed decay rate is represented by T2* and is always shorter than T2.

Image contrast is the difference in luminance or color that distinguishes one object from another in the same image. Contrast is created by using a selection of image acquisition parameters that weighs signals by T1, T2 or T2*, or no relaxation time. These images are known in the art as proton density images. For example, in the brain, T1-weighting (T1w) causes the nerve connections of white matter to appear white, and the congregations of neurons of gray matter to appear gray. Cerebrospinal fluid appears dark.

Contrast agents may be used to enhance tissue contrast in MRI images by inducing magnetic susceptibility contrast effects when injected. Magnetic susceptibility is a property of materials and is measure of the degree to which a material will become magnetized in an applied magnetic field. Materials may be classified into two groups based upon their individual susceptibility—paramagnetic materials and diamagnetic materials. Paramagnetic materials align with an applied magnetic field and are attracted to it. Diamagnetic materials on the other hand align against an applied magnetic field and are repelled by it. Accordingly, most commonly a paramagnetic contrast agent, typically a gadolinium compound, is employed to enhance MRI image contrast. As will be discussed in greater detail below, several different contrast enhancing agents may also be used.

Gadolinium-enhanced tissues and fluids appear extremely bright in weighted images, thereby providing high contrast sensitivity which facilitates the detection of vascular issues (tumors) and permits assessment of brain perfusion, such as that which occurs following a stroke. Cerebral blood volume (CBV) and cerebral blood flow (CBF) can also be measured, and other hemodynamic and vascular parameters can be derived from these measurements. However, a significant problem associated with the use of gadolinium-based contrast agents is their migration or leakage from a patient's blood vessels, This leakage results in undesirable T1 and T2 relaxation effects that significantly complicate the ability of treating physicians to evaluate T1-weighted images.

Image-based techniques have been developed to determine the effects of brain tumors and to address the problem of proper assessment of patients suffering from brain abnormalities. These techniques are integral to the management of patients with brain tumors and other neuro abnormalities and critical for the evaluation of new treatments in clinical trials. The standard approach requires a visual assessment of the contrast-agent enhancement (T1w+contrast agent) of a region of interest (ROI) in a magnetic resonance image (MRI). As shown in FIG. 1, evaluation of conventional pre- and post-contrast T1-weighted images 10 and 15 respectively is problematic since the complicated nature of tumors, especially treated tumors, coupled with the subjective and time-consuming methods of assessment creates an unacceptably high level of inconsistent interpretations. Consequently, even with expert readers (neuro radiologists), the inter-reader agreement on image interpretation is usually no better than 50-60%. Poor interpretation agreement is a significant limitation for clinicians as well as for use in multi-center clinical trials. Therefore, monitoring patients with brain tumors simply by visually comparing differences in enhancement has become increasingly more difficult.

Moreover, the development of automated methods to determine regions of interest on T1+C ROI images has several limitations. Unlike x-ray angiography (XA) or computed tomography (CT), the pixel values for the same tissue type on MR images vary widely due to a number of factors, even for the same sequence type. Some non-contrast enhancing tissues, such as blood products, can also appear bright, thus further confounding interpretation. Visual comparison of pre- and post-contrast images or subtraction of a pre-contrast image from the post-contrast image can compensate for these limitations, inasmuch as the process highlights areas of contrast enhancement. Nonetheless, as illustrated in FIGS. 2.A and 2.B, even on the basic subtraction image or simple difference map 5, the variability of the MR pixel values as well as image nonuniformity and/or variations arising from slight differences in acquisition methods, can lead to numerous regions such as region 7 having a bright appearance without contrast enhancement. Region 7 is barely visible in the post contrast image 9 of FIG. 2.A. Accordingly, the identification of truly enhanced tumors is at best challenging, and the automation of this process near impossible.

Prior art attempts to address the above-referenced problems include the following published works:

The first Nyu'l paper showing standardized mapping of image intensities (no difference map generation, no application of the Difference color look up table [CLUT]):

Nyu'l L. G., Udupa J. K., Zhang, X. New variants of a method of MRI scale standardization. IEEE Trans Med Imaging 2000; 19:143-50 Cross Ref Medline.

The first report of the approach to standardize and subtract:

Jensen T. R., Schmainda, K. M. Computer-aided detection of brain tumor invasion using must parametric MRI. Magn Reson Imaging 2009; 30:481-89.

The first initial disclosure on the Delta T1 mapping process (using standardization, subtraction, Difference CLUT) was in 2010 (ISMRM):

Bedekar, D, Jensen, T., Rand, S., et al., Delta T1 method: an automatic post-contrast ROI selection technique for brain tumors. In: Proceedings of the Annual Meeting of the international Society for Magnetic Resonance in Medicine, Stockholm, Sweden May 1-7, 2010.

A method that uses a Gaussian normalization approach to pre-process the pre- end post-contrast T1-weighted images before subtraction; the Difference CLUT is not used:

Ellingson, B. M., Kim, H. J., Woodworth, D. C., et al., Recurrent glioblastoma treated with bevacizumab: contrast-enhanced T1-weighted subtraction maps improve tumor delineation and aid prediction of survival in a multicenter clinical trial. Radiology 2014; 271:200-10.

An application of the dT1 to clinical trial data, showing it can provide equivalent and/or superior results compared to the current method of using central expert feeders;

Schmainda, K. M., Prah M. A., Zhang, Z., Snyder, B. S., Rand, S. D., Jensen, T. R. Barboriak, D. P., Boxerman, J. L., Quantitative delta T1 (dT1) as a replacement for adjudicated central reader analysis of contrast-enhancing tumor burden: A sub analysis of the American College of Radiology imaging Network 6677/Radiation Therapy Oncology Group 0625 Multicenter Brain Tumor Trial. AMR Am. J. Neuroradiol. 2019 40(7):1132-1139.

While simple “difference” maps are known in the art, they are known to be of relative (not quantitative) in value, do not account for confounding factors (such as post-surgical blood products), and are not able to identify subtle regions of true enhancement. Other prior art methods do not produce resulting maps on a consistent quantitative scale, cannot be generated automatically, do not routinely use the red/blue FLOW-CLUT, and have not demonstrated the ability to predict response to tumor recurrence such as illustrated in FIG. 4.

In view of the foregoing, it is apparent to those skilled in the art that a need exists for a system and a method for generating delta T1 maps that may be used to quantify levels of contrast enhancement by subtracting pre- from post-contrast T1-weighted images, each of which were first transformed to a quantitative scale.

### SUMMARY OF THE INVENTION

A system and a method for generating and displaying quantitative delta T1-weighted images or maps which are displayed using a “FLOW” (red/blue) color look up table (CLUT).

In an embodiment, the output is a quantitative difference (delta T1 or dT1) map that makes possible the objective identification (i.e., automation) of a region of interest across time and patients. The delta T1 maps have the further benefits of eliminating factors (such as post-surgical or treatment-related blood products) that can confound the current practice of subjectively evaluating T1-weighted images and can manifest subtle regions of true enhancement not otherwise visible.

In another embodiment, a method is disclosed for generating delta T1-weighted maps automatically without any additional processing steps or manual intervention.

In yet another embodiment, a method is provided which employs empirically determined cutoff thresholds that delineate enhancing and non-enhancing regions in the delta T1-weighted images.

In still another embodiment a method is disclosed for generating delta T1-weighted maps which employs a commonly available FLOW-CLUT (red-blue) to provide rapid and objective visualization via red image enhancement.

In another embodiment, a method is disclosed for generating delta T1-weighted maps automatically which computationally retains small values of “blue” (non-enhancing) regions that provide the user with easy spatial visualization of underlying anatomy.

In yet another embodiment, a method is disclosed for generating delta T1-weighted maps automatically that are repeatable and consistent over time.

Other advantages of the present invention will be readily apparent to one skilled in the art from the description, the figures, and the appended claim.

### BRIEF DESCRIPTION OF THE DRAWINGS

For a more complete understanding of the present disclosure and its advantages, reference is now made to the following description taken in conjunction with the accompanying drawings. The patent application file contains at least one drawing executed in color. Copies of this patent or patent application with color drawing(s) will be provided by the Office upon request and payment of the necessary

FIG. 1.A illustrates a conventional pre-contrast T1-weighted image;

FIG. 1.B illustrates a conventional post-contrast T1-weighted image;

FIG. 2.A depicts a post-contrast T1-weighted image;

FIG. 2.B illustrates a simple difference map without standardization:

FIG. 3.A illustrates the quantitative properties of a delta T1 image;

FIG. 3.B illustrates the delineation between a T1 image and a T1 post contrast image (T1+C).

FIG. 3.C illustrates the difference between the T1 and T1+C images of FIGS. 3.A and 3.B and a delta T1 image map created in accordance with an embodiment of the present invention;

FIG. 4.A illustrates a T1 map image of a non-invasive tumor;

FIG. 4.B illustrates a T1 map showing the effect of treatment of the non-invasive tumor illustrated in FIG. 4.A;

FIG. 4.C illustrates a delta T1 map's ability to enhance the response of he non-invasive tumor of FIGS. 4.A and 4.B to treatment;

FIG. 5.A illustrates the step of co-registration of a pre-contrast T1 image and a post-contrast T1+C weighted image in accordance with an embodiment;

FIG. 5.B illustrates the step of standardization or calibration of the co-registered pre- and post-contrast images created in the step of FIG. 5.A in accordance with an embodiment;

FIG. 5.C illustrates the standardized or calibrated pre- and post-contrast images created in the step of FIG. 5.B in accordance with an embodiment;

FIG. 5.D illustrates the step of subtracting the standardized co-registered pre-contrast T1 image from the standardized post-contrast T1+C image to create a delta T1 image or map (dT1) in accordance with an embodiment;

FIG. 5.E illustrates the step of applying a FLOW color look up table (CLUT) to a delta T1 map created in the step of FIG. 5.D to generate a red/blue image having true contrast enhancement in accordance with an embodiment;

FIG. 6 is an image of the standardization step of FIG. 5.B enlarged to more clearly illustrate the elements thereof; and

FIG. 7.A illustrates examples of T1 weighted images of three patients before standardization; and

FIG. 7.B illustrates examples of T1 weighted images of the three patients of FIG. 7.A after standardization.

### DETAILED DESCRIPTION OF THE INVENTION

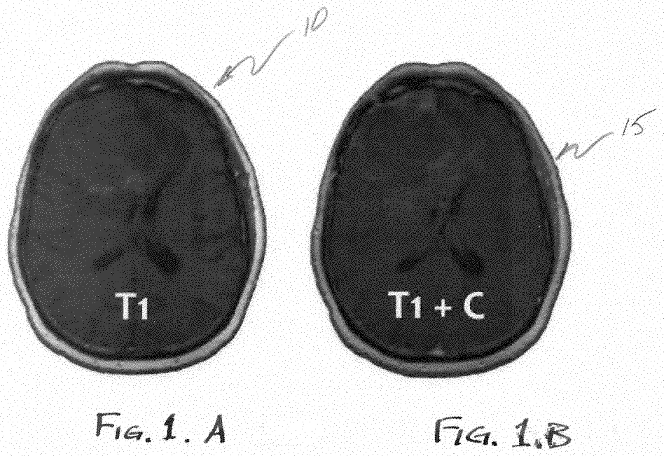

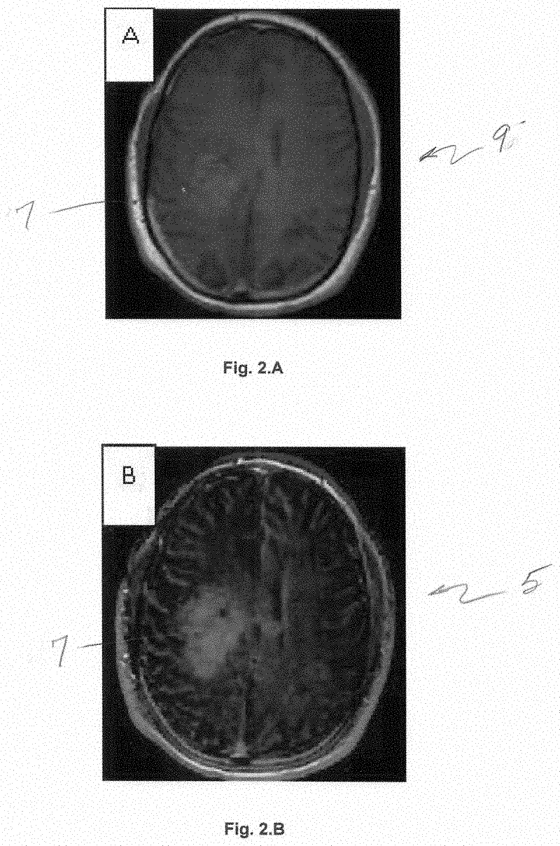

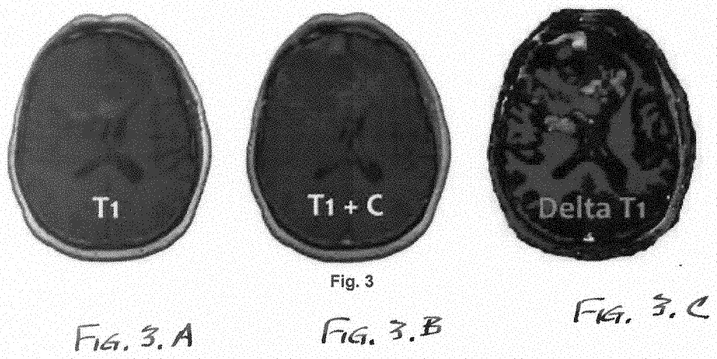

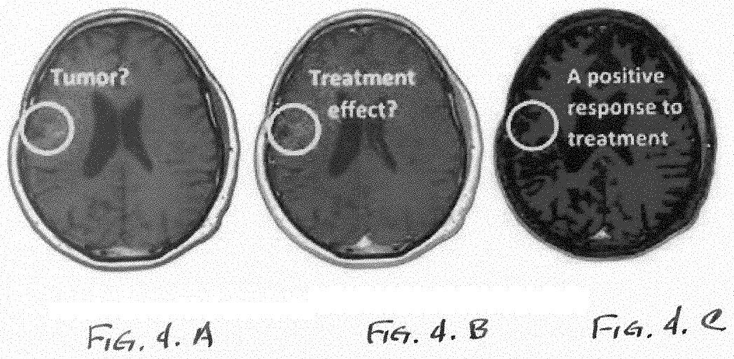

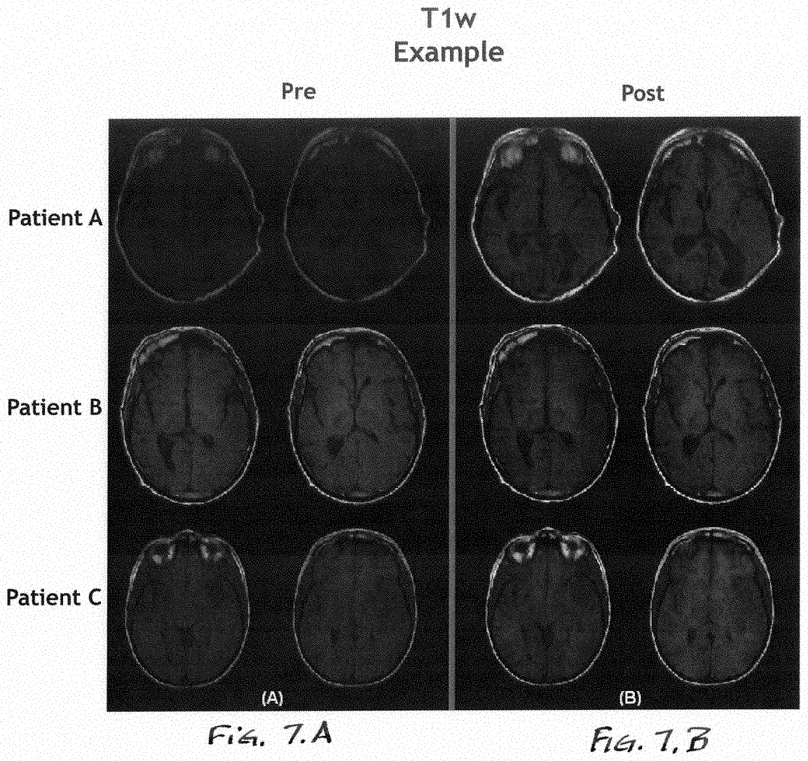

FIGS. 1 through 7, discussed below, and the various embodiments used to describe the principles of the present disclosure are by way of illustration only and should not be construed in any way to limit the scope of the disclosure. Those skilled in the art will understand that the novel principles, elements, and methodologies of the present invention may be implemented in any suitably arranged and configured delta T1-weighted image generating system without departing from the scope of the invention. In the following description, detailed descriptions of well-known functions, configurations, or systems will be omitted since they would unnecessarily obscure the subject matter of the various embodiments of the present disclosure. Terms used herein must be understood based on the descriptions made herein.

### The Method of the Present Invention

The method of generating and displaying quantitative T1-weighted subtraction maps, also referred to herein as delta T1 maps, includes the following steps:

1. Pre- and post-contrast T1-weighted images 10 and 15 respectively as shown in FIGS. 1.A and 1.B of a selected portion of a patient's anatomy are created using magnetic resonance imaging (MRI) technology and procedures. By way of example and not of limitation, the images shown herein are of a patient's brain; however, is to be understood that images of other anatomical elements may be used without departing from the scope of the present invention.

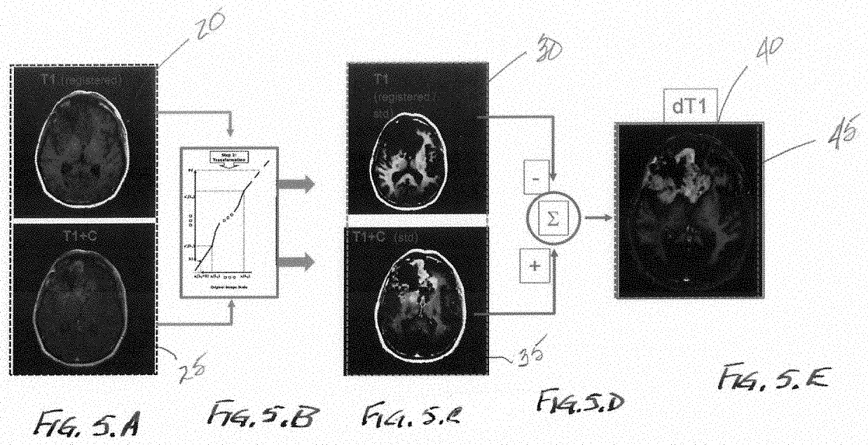

2. Both images are co-registered to each other to a consistent quantitative scale using commonly available image co-registration algorithms incorporating a fixed set of pre- and post-contrast images on a voxel by voxel basis as illustrated at 20 and 25 in FIG. 5.A.

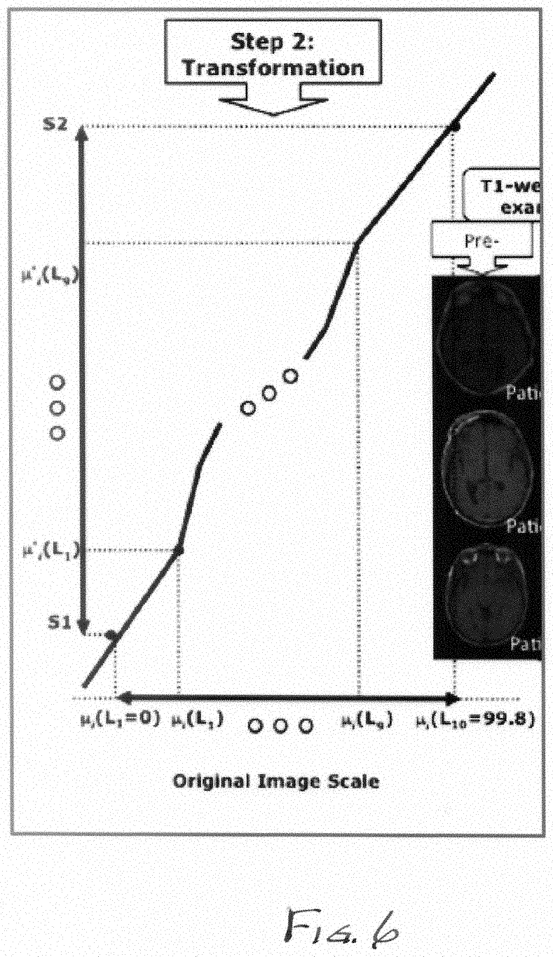

3. Pre- and post-contrast T1-weighted images are then standardized or calibrated as shown in FIG. 5.B (enlarged for clarity in FIG. 6) using the technique developed by Nyu'l et. al. to obtain a standardized T1 (stdT1) image identified at 30 in FIG. 5.C and a standardized T1+C (stdT1+C) image identified at 35 in FIG. 5.C and corresponding datasets associated with each image. The technique developed by Nyu'l et al. is published in an article appearing in Magnetic Resonance in Medicine, the entire contents of which are incorporated herein by reference to the maximum extent allowable by law, Nyu'l et al., On Standardizing the MR Image Intensity Scale, Magnetic Resonance in Medicine 42:1072-1081 (1999)).

4. At step 4, illustrated in FIG. 5.D, the standardized T1 dataset associated with the image 30 is subtracted from the standardized T1+C dataset associated with the image 35 to generate one or more delta T1 maps (dT1) via a subtraction of pairs of voxel values between co-registered and post-contrast anatomical images that is performed throughout the image volume.

5. An empirically determined threshold (3000 or higher on the standardized scale) is applied to each of the one or more delta T1 maps to obtain an enhancing lesion region of interest (ROI) illustrated at 40 in the dT1image 45 in FIG. 5.E. The “threshold” is defined as the magnitude of the scale value which distinguishes between mage enhancing (a value higher than the empirically determined threshold) and image non-enhancing, (a value lower than the threshold) on the standardized scale. Since the delta T1 maps are quantitative, this threshold step can be automated using Artificial intelligence (AI).

6. Noisy voxels mainly around skull and eyes area can be manually removed.

7. As shown in FIG. 5.E, a Difference or a FLOW (blue-red) color look up table (CLUT) is applied to provide rapid and objective visual determination of true color enhancing regions. The CLUT may be applied automatically without manual intervention or assistance to render the red/blue image as shown, The red represents regions of “True” contrast enhancement as the subtraction step removes confounding factors that cause bright signal on the pre-contrast T1 weighted images, such as post-surgical blood products. The dT1 maps also enable detection of subtle contrast enhancing regions that may not be clearly visible on the T1+C images.

Imperfections in the image acquisition process can result in image intensity variations such that the same tissue in different locations have different image intensities. These image artifacts are referred to as bias field, shading, intensity inhomogeneity, or intensity nonuniformity. Although the bias field is often difficult to observe by the human eye, it can degrade the accuracy of automated analysis techniques significantly. Accordingly, a number of techniques or methods have been proposed to correct the bias field in medical images, and the method of creating dT1 maps in accordance with the instant invention optionally, intermediate step 1 and step 2, includes a bias correcting step that may be performed using a commonly available algorithm (e.g., N4ITK). However, this step may be omitted without departing from he scope of the present invention.

### The System of the Present Invention

The process defined in the above steps can be automated in repeatable and consistent image processing algorithm, requiring a few seconds (<10 seconds) of processing time.

The novel methodology herein disclosed is a software application that can be integrated into MRI imaging platforms as a pre-processing step for neuro assessment. The process steps may be stored on a non-transitory computer-readable storage medium or hardware such as a disc, a removable hard drive, or a thumb drive and the like adapted to store non-transitory process instructions that, when executed by a computer, cause it to perform the process steps herein disclosed. The application identifies the pre- and post-contrast T1 weighted images automatically, performs the above processing steps, and automatically generates a map that can be used within any DICOM (Digital Information and Communication in Medicine) medical imaging platform.

Changes may be made in the above methods and systems without departing from the scope hereof. It should be noted that the matter contained in the above description and/or shown in the accompanying figures should be interpreted as illustrative and not in a limiting sense. The following claims are intended to cover all generic and specific features described herein, as well as all statements of the scope of the present systems.

What is claimed is:

1. A method for generating and displaying quantitative T1-weighted magnetic resonance subtraction map images comprising:
creating pre- and post-contrast T1-weighted images of a selected portion of a patient's anatomy using magnetic resonance imaging (MRI) technology and procedures;
co-registering the pre- and post-contrast T1-weighted images to each other to a consistent quantitative scale using commonly available image co-registration algorithms;
standardizing or calibrating the co-registered pre- and post-contrast T1-weighted images using commonly available standardizing techniques to obtain a standardized T1 (stdT1) image and a standardized T1+C (stdT1+C) image and associated datasets;
subtracting the standardized T1 dataset from the standardized T1+C dataset to generate one or more delta T1 maps (dT1);
applying an empirically determined threshold to each of the one or more delta T1 maps to obtain an enhancing lesion region of interest (ROI); and
applying a preselected color look up table (CLUT) to each of the one or more delta T1 maps whereby rapid and objective visual determination of true color enhancing regions is obtained.

2. The method of claim 1 further including a bias-correcting step intermediate the co-registration step and the standardization step whereby image intensity variations such as bias field, shading, intensity inhomogeneity, or intensity nonuniformity are removed.

3. The method of claim 2 wherein the bias-correcting step may be performed using a commonly available algorithm.

4. The method of claim 3 wherein the commonly available algorithm comprises the N4ITK algorithm.

5. The method of claim 1 wherein the commonly available image co-registration algorithms incorporate a fixed set of pre- and post-contrast images on a voxel by voxel basis.

6. The method of claim 1 wherein the step of subtracting the standardized T1 dataset from the standardized T1+C dataset comprises subtracting pairs of voxel values between co-registered and post-contrast anatomical images that is performed throughout the image volume.

7. The method of claim 1 wherein the step of applying an empirically determined threshold is automated using Artificial Intelligence (AI).

8. The method of claim 1 further including the step of removing noisy voxels around the skull and eyes area.

9. The method of claim 8 wherein the noisy voxels around the skull and eyes area are removed manually.

10. A non-transitory computer-readable storage medium or hardware having data stored therein representing software executable by a computer, the software including non-transitory process instructions that, when executed by a computer, cause it to perform the process steps of:
creating pre- and post-contrast T1-weighted images of a selected portion of a patient's anatomy using magnetic resonance imaging (MRI) technology and procedures;
co-registering the pre- and post-contrast T1-weighted images to each other to a consistent quantitative scale using commonly available image co-registration algorithms;
standardizing or calibrating the co-registered pre- and post-contrast T1-weighted images using commonly available standardizing techniques to obtain a standardized T1 (stdT1) image and a standardized T1+C (stdT1+C) image and associated datasets;
subtracting the standardized T1 dataset from the standardized T1+C dataset to generate one or more delta T1 maps (dT1);
applying an empirically determined threshold to each of the one or more delta T1 maps to obtain an enhancing lesion region of interest (ROI); and
applying a preselected color look up table (CLUT) to each of the one or more delta T1 maps whereby rapid and objective visual determination of true color enhancing regions is obtained.
